(19) United States
(12) Patent Application Publication
(10) Pub. No.: US 2024/0377353 A1
(43) Pub. Date: Nov. 14, 2024

(54) CIRCUITRY FOR MEASUREMENT OF ELECTROCHEMICAL CELLS

(71) Applicant: Cirrus Logic International Semiconductor Ltd., Edinburgh (GB)

(72) Inventors: John P. LESSO, Edinburgh (GB); James T. DEAS, Edinburgh (GB); Cedric ANDRIEU, Edinburgh (GB)

(73) Assignee: Cirrus Logic International Semiconductor Ltd., Edinburgh (GB)

(21) Appl. No.: 18/650,430

(22) Filed: Apr. 30, 2024

Related U.S. Application Data
(60) Provisional application No. 63/501,020, filed on May 9, 2023.
(60) Provisional application No. 63/512,233, filed on Jul. 6, 2023.
(60) Provisional application No. 63/592,275, filed on Oct. 23, 2023.

(51) Int. Cl. G01N 27/416

(52) U.S. Cl. CPC G01N 27/416

(57) ABSTRACT

Circuitry for processing an analyte signal obtained from an electrochemical cell, the circuitry comprising: a first input node for receiving the analyte signal; measurement circuitry having a first signal path coupled to the first input node, the measurement circuitry configured to output an output signal based on the analyte signal, the measurement circuitry comprising a first circuit element having a first impedance, the first circuit element coupled to the first signal path; and control circuitry configured to reset one or more first voltages in the first signal path of the measurement circuitry to a respective first reference value.

### TECHNICAL FIELD

The present disclosure relates to circuitry for measuring characteristics in electrochemical cells.

### BACKGROUND

Electrochemical sensors are widely used for the detection or characterisation of one or more particular chemical species, analytes, typically as an oxidation or reduction current. Such sensors comprise an electrochemical cell, consisting of two or more electrodes configured for contact with an analyte whose concentration is to be ascertained.

For potentiostatic measurement, sensors may also comprise circuitry for driving one or more of the electrodes and for measuring a response signal at one or more of the electrodes. The measured response signal can be processed to determine a concentration of an analyte.

For potentiometric measurement, a potential difference is measured between an electrode and an analyte with no external bias and with no current flow. A working electrode (indicator electrode) of the electrochemical cell can be used as a proxy for the electrode, and a reference electrode can be used as a proxy for the analyte. Thus, the potential difference between the working electrode and the reference electrode gives an indication of a property of the electrode and the analyte. To ensure little or no current flows from the electrochemical cell, a high input impedance to any measurement circuitry is advantageous. Synthesizing such high input impedance can often require either active circuitry or complex process options which can lead to added cost and complexity.

### SUMMARY

According to a first aspect of the disclosure, there is provided circuitry for processing an analyte signal obtained from an electrochemical cell, the circuitry comprising: a first input node for receiving the analyte signal; measurement circuitry having a first signal path coupled to the first input node, the measurement circuitry configured to output an output signal based on the analyte signal, the measurement circuitry comprising a first circuit element having a first impedance, the first circuit element coupled to the first signal path; and control circuitry configured to reset one or more first voltages in the first signal path of the measurement circuitry to a respective first reference value.

The control circuitry may be configured to reset the one or more voltages in the signal path based on the output signal.

The output signal may be a differential output signal. The control circuitry may be configured to reset the one or more voltages in the signal path based on a common mode of the differential output signal.

The control circuitry may be configured to reset the one or more voltages in the signal path in response to the common mode of the differential output signal exceeding a threshold common mode level.

The one or more first voltages may comprise a plurality of first voltages at different nodes of the first signal path. The control circuitry may be configured to reset each of the plurality of first voltages sequentially.

The circuitry may comprise a second input node. The analyte signal may be received between the first and second input nodes. The measurement circuitry may further comprise: a second signal path coupled to the second input node; and a second circuit element having a second impedance. The second circuit element may be coupled to the second signal path. The control circuitry may be configured to reset one or more second voltages in the second signal path to a respective second reference value.

The control circuitry may be configured to sequentially reset the one or more first voltages and the one or more second voltages. Alternatively, the control circuitry may be configured to simultaneously reset the one or more first voltages and the one or more second voltages.

The circuitry may further comprise processing circuitry configured to: sample the output signal during a first sampling period after reset of the respective voltage to generate a sampled output signal.

The processing circuitry may comprise an analog-to-digital converter (ADC). The ADC may comprise a sigma-delta ADC, such as an incremental sigma-delta ADC. Simultaneous to resetting the one or more voltages in the signal path, the control circuitry may be configured to reset the ADC.

The processing circuitry may be configured to: determine a characteristic of the analyte signal based on the sampled output signal.

The processing circuitry may be configured to: obtain a plurality of samples of the output signal during the first sampling period; fit the plurality of samples to a predetermined curve; and determine the characteristic of the analyte signal based on the predetermined curve.

The processing circuitry may be configured to: apply compensation to the sampled output signal to compensate for a response of the measurement circuitry to the reset of the one or more first voltages in the first signal path.

The processing circuitry may be configured to apply compensation to the sampled output signal to compensate for a filtering characteristic of measurement circuitry.

The processing circuitry may be configured to apply an inverse transfer function to the sampled output signal. The inverse transfer function may be inverse to a transfer function of the filtering characteristic of the measurement circuitry.

The processing circuitry may be configured to apply compensation to the sampled output signal to compensate for a response of the electrochemical cell to the reset of the one or more first voltages.

The first circuit element may comprise an input filter configured to filter the analyte signal to generate a filtered analyte signal.

Reset of the one or more first voltages may comprise resetting charge on a capacitor of the input filter to a known charge.

The first sampling period may be shorter than a settling time of the input filter.

The measurement circuitry may comprise a gain stage configured to apply a gain to the filtered analyte signal to obtain the output signal.

The first circuit element may comprise a first input filter configured to filter the analyte signal to generate a first filtered analyte signal and a first gain stage configured to apply a first gain to the first filtered analyte signal to obtain a first output signal. The second circuit element may comprise a second input filter configured to filter the analyte signal to generate a second filtered analyte signal and a second gain stage configured to apply a second gain to the second filtered analyte signal to obtain a second output signal. The first and second output signals may be combined to obtain a combined output signal.

The first and second gain stages are biased in dependence on respective first and second output signals.

The circuitry may further comprise: an analog-to-digital converter (ADC) configured to sample the combined output signal to obtain a sampled output signal; and compensation circuitry configured to apply compensation to the sampled output signal based on filter characteristics of the first and second input filters.

The ADC may comprise an incremental sigma-delta ADC. The control circuitry may be configured to reset the ADC when resetting the one or more first voltages in the first signal path.

The circuitry may further comprise a first ADC configured to sample the first output signal to obtain a first sampled output signal; a second ADC configured to sample the second output signal to obtain a second sampled output signal; compensation circuitry configured to: apply compensation to the first sampled output signal based on a first filter characteristic of the first filter; and apply compensation to the second sampled output signal based on a second filter characteristic of the second filter.

The circuit element may comprise one or more diodes. The one or more diodes may comprise silicon diodes or polysilicon diodes. For example, the one or more diodes may comprise first and second diodes coupled in series, and third and fourth diodes coupled in series. Optionally a first coupling between the first and second diodes may be coupled to a second coupling between the third and fourth diodes.

The control circuitry may comprise a digital signal processor (DSP).

According to another aspect of the disclosure, there is provided circuitry for processing an analyte signal obtained from an electrochemical cell, the circuitry comprising: a first input node for receiving the analyte signal; measurement circuitry having a first signal path coupled to the first input node, the measurement circuitry configured to output an output signal based on the analyte signal, the measurement circuitry comprising a first impedance comprising a polysilicon diode coupled to the first signal path; and control circuitry configured to reset one or more first voltages in the first signal path of the measurement circuitry to a respective first reference value.

According to another aspect of the disclosure, there is provided an integrated circuit (IC), comprising: circuitry as described above.

According to another aspect of the disclosure, there is provided an integrated circuit (IC), comprising: first channel circuitry for processing a first analyte signal from a first electrochemical sensor; and second channel circuitry for processing a second analyte signal from a second electrochemical sensor, wherein the first and second channels each comprise circuitry as described above.

According to another aspect of the disclosure, there is provided a multi-analyte sensor, comprising: an IC as described above, the first electrochemical sensor; and the second electrochemical sensor.

According to another aspect of the disclosure, there is provided a system comprising: circuitry as described above, and the electrochemical cell.

The electrochemical cell may comprise a counter electrode and a first working electrode. The electrochemical cell may further comprise a second working electrode.

The electrochemical cell may comprise an anode and a cathode, wherein the first electrode is the cathode.

According to another aspect of the disclosure, there is provided an electronic device, comprising circuitry, an IC, or a system as described above.

The electronic device may comprise one of an analyte monitoring device or an analyte sensing device, a battery, a battery monitoring device, a mobile computing device, a laptop computer, a tablet computer, a games console, a remote control device, a home automation controller or a domestic appliance, a toy, a robot, an audio player, a video player, or a mobile telephone, and a smartphone.

The electronic device may comprise one of an analyte monitoring device or an analyte sensing device, a battery, a battery monitoring device, a mobile computing device, a laptop computer, a tablet computer, a games console, a remote control device, a home automation controller or a domestic appliance, a toy, a robot, an audio player, a video player, or a mobile telephone, and a smartphone.

Throughout this specification the word “comprises”, or variations such as “comprises” or “comprising”, will be understood to imply the inclusion of a stated element, integer or step, or group of elements, integers, or steps, but not the exclusion of any other element, integer or step, or group of elements, integers or steps.

### BRIEF DESCRIPTION OF DRAWINGS

Embodiments of the present disclosure will now be described by way of non-limiting examples with reference to the drawings, in which:

FIG. 1 illustrates a schematic diagram and electrical equivalent circuit for a three-electrode electrochemical cell;

FIG. 2 illustrates a schematic diagram and electrical equivalent circuit for a two-electrode electrochemical cell;

FIG. 3 illustrates a schematic diagram of a potentiometric sensor;

FIG. 4 is a schematic diagram of a known high input impedance measurement circuit; and

FIG. 5 is a schematic diagram of a measurement circuit according to embodiments of the present disclosure;

FIG. 6 is a flow diagram of a process which may be implemented by the measurement circuit of FIG. 5;

FIG. 7 is a schematic diagram of a measurement circuit according to embodiments of the present disclosure;

FIG. 8 is a graphical representation of voltages in the measurement circuit of FIG. 7;

FIG. 9 is an example implementation of control circuitry of the measurement circuit of FIG. 7;

FIGS. 10 to 13 are schematic diagrams of measurement circuits according to embodiments of the present disclosure; and

FIG. 14 is a schematic diagram of an example impedance.

### DESCRIPTION OF EMBODIMENTS

Embodiments of the present disclosure relate to the measurement of signals (such as analyte signals) in electrochemical cells. In particular, embodiments relate to improved methods and circuitry for the characterisation of electrochemical cells using potentiometric measurements.

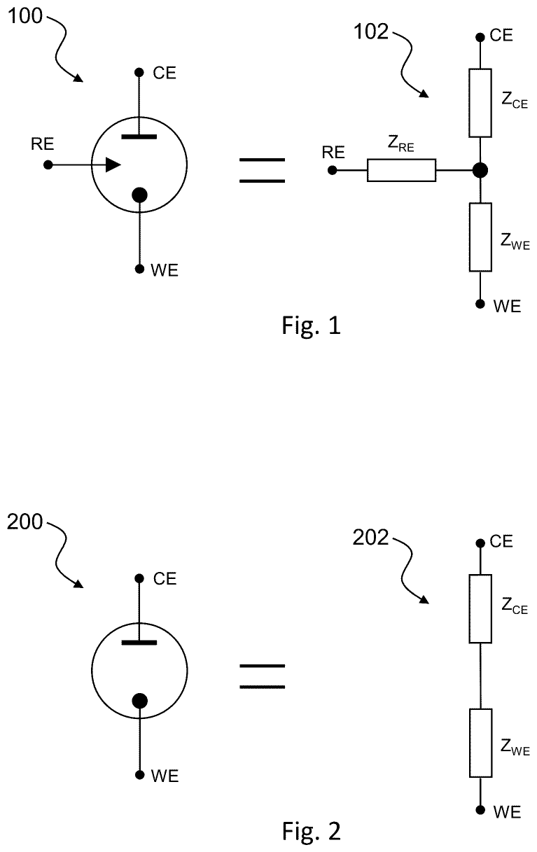

FIG. 1 is a schematic diagram of an example electrochemical cell 100 comprising three electrodes, namely a counter electrode CE, a working electrode WE and a reference electrode RE. FIG. 1 also shows an equivalent circuit 102 for the electrochemical cell 100 comprising a counter electrode impedance ZCE, a working electrode impedance ZWE and a reference electrode impedance ZRE.

FIG. 2 is a schematic diagram of another example electrochemical cell 200 comprising two electrodes, namely a counter electrode CE and a working electrode WE. The electrochemical cell 200 varies for the cell 100 with the omission of the reference electrode RE. FIG. 2 also shows an equivalent circuit 102 for the electrochemical cell 200 comprising a counter electrode impedance ZCE and a working electrode impedance ZWE.

In some embodiments, the working electrode WE comprise an assay or chemical of interest. For example for the analysis of glucose as an analyte, the working electrode may comprise a layer of glucose oxidase. The counter electrode CE is provided to form an electrical or ohmic connection with the working electrode WE. Optionally, the reference electrode is provided, which is typically a sensing point between the working electrode WE and the counter electrode CE, allowing independent measurement of the potential associated with each of the working and counter electrodes WE. CE, rather than just measuring a potential difference between the counter and working electrodes CE, WE.

Embodiments of the present disclosure will be described with reference to these example electrochemical cells 100, 200. It will be appreciated, however, that the techniques and apparatus described herein may be used in conjunction with any conceivable electrochemical system, including but not limited to electrochemical cells comprising at least two electrodes (e.g. a counter electrode CE, a working electrode WE and optionally a reference electrode RE), or electrochemical cells with more than three electrodes (e.g. two or more counter electrodes and/or two or more working electrodes). Electrodes of the electrochemical cells described herein may also be referred to as anodes and/or cathodes as is conventional in the field of electrical batteries.

The cells 100, 200 may be implemented for potentiometric measurement or potentiostatic measurement.

In potentiostatic arrangements, to determine a characteristic of either of the electrochemical cells 100, 200, and therefore an analyte concentration, it is conventional to apply a bias voltage at the counter electrode CE and measure a current at the working electrode WE. When provided, the reference electrode RE may be used to measure a voltage drop between the working electrode WE and the reference electrode RE. The bias voltage is then adjusted to maintain the voltage drop between the reference and working electrodes RE, WE constant. As the resistance in the cell 100 increases, the current measured at the working electrode WE decreases. Likewise, as the resistance in the cell 100 decreases, the current measured at the working electrode WE increases. Thus the electrochemical cell 100 reaches a state of equilibrium where the voltage drop between the reference electrode RE and the working electrode WE is maintained constant. Since the bias voltage at the counter electrode CE and the measured current at WE are known, the resistance of the cell 100 can be ascertained.

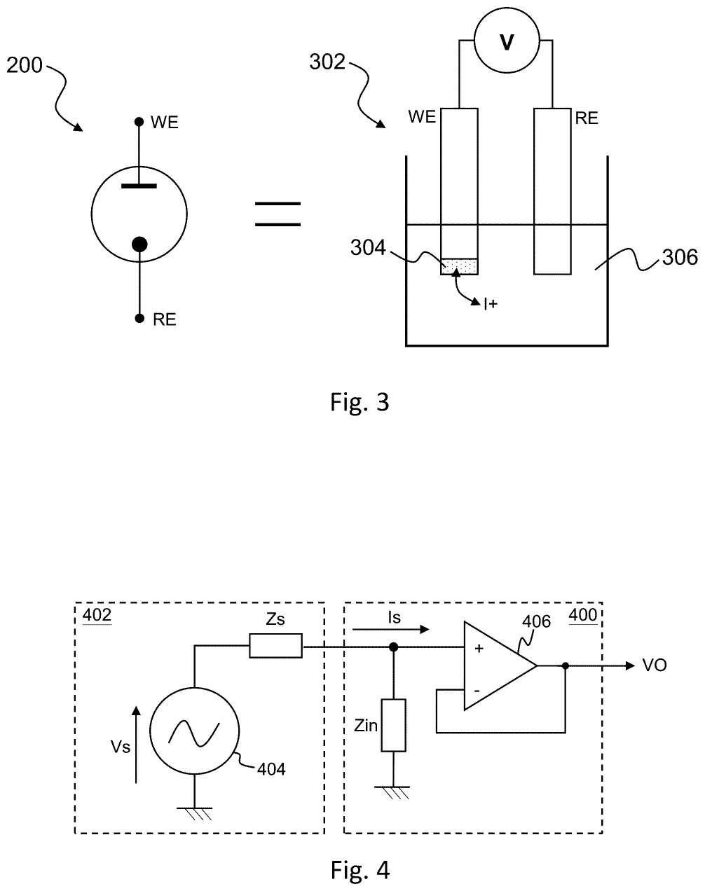

When the cells 100, 200 are configured for potentiometric sensing, a potential across the cells 100, 200 may be measured without applying any bias voltage to the cells 100, 200. As an example, FIG. 3 illustrates the electrochemical cell 200 of FIG. 2 alongside a schematic diagram of an example implementation of the electrochemical cell 200 as a potentiometric sensor. The working electrode WE comprises an ion-selective membrane 304, which may be configured to uptake only a specific ion (in this case the cation, I+) from an electrolyte solution 306. As such, the potential difference between the working electrode WE and the reference electrode RE depends on the concentration of that particular ion analyte in the electrolyte solution 306.

To accurately measure the potential difference across the cell 200, as little as possible current (ideally no current) need flow into the cell 200. Hence, a typical approach to voltage measurement is to couple each of the working and reference electrodes WE, RE to high input impedance buffers which are used, in turn, to drive one or more ADCs (e.g. two single ended ADCs or one differential ADC). A digital output signal is then derived which represents the potential difference between working and reference electrode WE, RE of the cell 200.

FIG. 4 is a schematic diagram of a typical measurement circuit 400 for measuring a potential difference Vs across the two-electrode cell 200 implemented as a potentiometric sensor. An equivalent circuit model 402 for the cell 200 is shown in FIG. 4. The model comprises a voltage source 404 (generating the potential difference or sense voltage Vs) and a series impedance Zs coupled. The voltage source 404 is coupled between a reference voltage (in this case ground) and the series impedance Zs which itself is coupled to an input of the measurement circuit 400. The measurement circuit 400 comprises a buffer amplifier 406 and an input impedance Zin. A non-inverting input of the buffer amplifier 406 is coupled to the series impedance Zs of the cell 200. The input impedance Zin is coupled between the non-inverting input of the buffer amplifier 406 and a reference voltage (in this case ground). An inverting input and output of the buffer amplifier 406 are coupled together. Thus, the measurement circuit 400 is configured as a high input impedance buffer amplifier which buffers the sense voltage Vs across the cell 200 to the output of the measurement circuit 400.

To maximise signal gain, the input impedance Zin of the measurement circuit 400 is typically at least an order of magnitude higher than the series impedance Zs of the cell 200. With electrochemical sensors typically having an impedance in the gigaohm range (e.g. 1-10 GΩ), this can lead to the measurement circuit 400 having an input impedance Zs in the order of teraohms (e.g. 1-10TΩ). To operate at such high input impedance, the measurement circuit 400 is required to have low leakage. Such operation can lead to high power consumption and large circuit area. In addition, high input impedances are susceptible to noise and stray current which manifest in large drift and/or noise in the output voltage VO of the measurement circuit 400. This noise is undesirable particularly for potentiometric applications, since such sensors are relatively insensitive to changes in electrolyte or analyte concentration; a large change in concentration of an analyte or electrolyte in a potentiometric cell manifests in a relatively small change in sensor voltage Vs.

Embodiments of the present disclosure aim to address or at least ameliorate one or more of the above issues by accepting that capacitance may cause stray current to build up at a sensor in a measurement circuit. To avoid accumulation of leakage charge, embodiments of the present disclosure provide circuitry for periodically resetting one or more nodes of the measurement circuit to one or more known voltages. Then, during the settling time after such reset, an output of the measurement circuit can be measured to determine a response of the cell to the reset. Since at the time of reset the voltages at the respective reset nodes are known, the response of the sensor and circuit during that settling time can be processed to obtain an accurate representation of the sensor. Thus, an accurate measure of analyte or electrolyte concentration can be obtained.

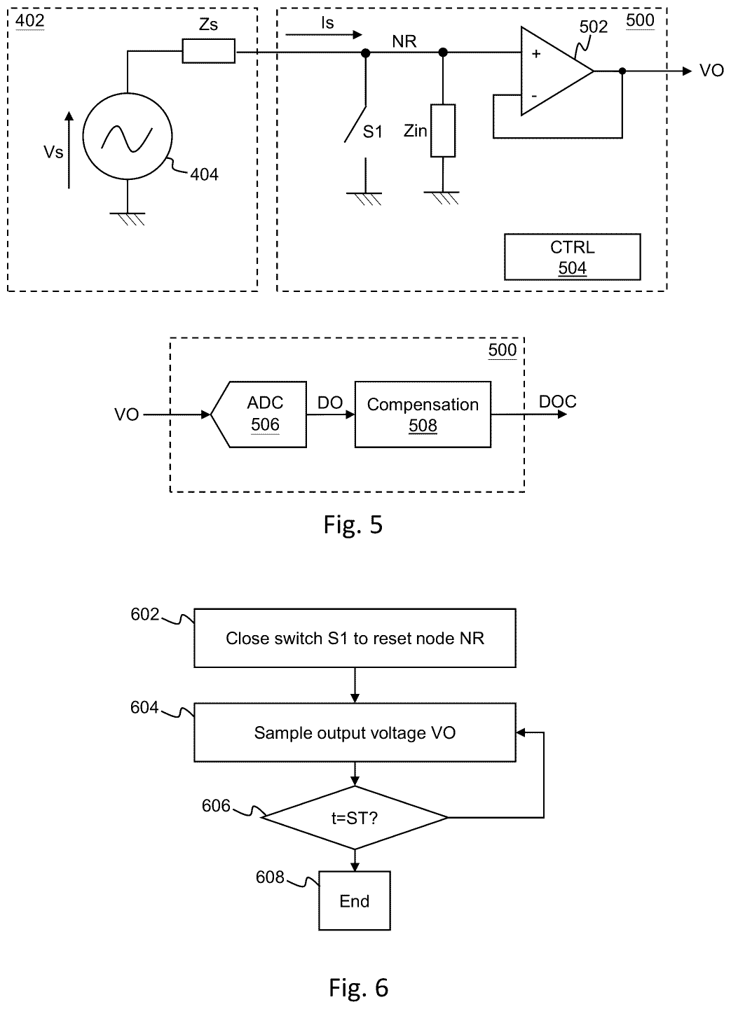

FIG. 5 is a schematic diagram of a measurement circuit 500 for measuring a potential difference Vs across the two-electrode cell 200 implemented as a potentiometric sensor. An equivalent circuit model 402 for the cell 200 is the same as that which is shown in FIG. 4. The measurement circuit 500 comprises an amplifier 502 and an input impedance Zin. A non-inverting input of the amplifier 502 is coupled to the series impedance Zs of the cell 200. The input impedance Zin is coupled between the non-inverting input of the amplifier 502 and a reference voltage (in this case ground). An inverting input and output of the amplifier 502 are coupled together. Thus, like the measurement circuit 400, the measurement circuit 500 is configured as a high input impedance buffer amplifier which buffers the sense voltage Vs across the cell 200 to the output of the measurement circuit 500 manifested as an output voltage VO. The input impedance

The measurement circuit 500 further comprises a reset switch S1 coupled at a reset node NR and the reference voltage (in this case ground). The reset node NR is coupled to the non-inverting input of the measurement circuit 500. Thus, when the reset switch S1 is closed, the input impedance Zin is bypassed, and a known reference voltage is applied at the reset node NR.

The measurement circuit 500 further comprises control circuitry 504 (i.e. a controller) configured to control the switch S1. The control circuitry 504 may be configured to control the switch S1 to reset the reset node NR periodically or in response to one or more external signals. For example, the reset node NR may be reset on determination of a noise condition in the output voltage VO, such as there being too much noise in the output voltage VO or a determination that the output voltage VO inaccurately approximates the sensor voltage Vs.

The measurement circuit 500 may further comprise further processing circuitry for sampling the output voltage VO. Such processing circuitry may comprise an analog-to-digital converter (ADC) 506. Such processing circuitry may additionally comprise compensation circuitry 508.

The ADC 506 may be a sigma delta ADC, such as an incremental sigma-delta ADC. The ADC 506 may be configured to sample the output voltage VO to generate a sampled output voltage DO.

The compensation circuitry 508 may comprise a digital signal processor. The compensation circuitry 508 may be configured to apply compensation to the sampled output voltage DO to compensate for the effect of settling after reset of the reset node NR. The compensation circuitry 508 may perform additional functions as will be described below with reference to further Figures.

FIG. 6 is as flow diagram 600 of an example process 600 which may be implemented by the measurement circuit 500.

At step 602, the control circuitry 504 may control the switch S1 to close, thus resetting the reset node NR to the reference voltage (e.g. ground), after which the switch S2 is opened. Optionally, if the ADC 506 is provided, the control circuitry 504 may also reset the ADC 506. The sample signal provided to close the switch S1 may be provided to the ADC 506 to reset the ADC. This is particularly the case where the ADC is implemented as a sigma-delta ADC, such as an incremental sigma-delta ADC (ISD ADC). In the example of an ISD ADC, the internal state of the ISD ADC is reset between measurement to ensure that the previous state of the ISD ADC (associated with a previous measurement) does not affect the current measurement.

At step 604, during a predetermined sampling period ST, the output voltage VO may be sampled by the ADC 506 to obtain a sampled output voltage DO. The sampling period ST may be chosen so as to approximately two orders of magnitude less than the settling time of the impedance Zin. For example, if the settling time of the impedance Zin is in the order of seconds, the sampling period ST may be in the order of milliseconds. It will be appreciated that the effect of drift will be smallest at the point of reset. However, the longer the sampling period ST, the less noise will affect the sampled signal. Thus, a longer sampling period ST may result in increased integration drift but decreased sensitivity to noise. Conversely, a shorter sampling period ST may result in decreased integration drift but increased sensitivity to noise. The sampling period ST may be selected to trade-off the effect of offset drift versus signal noise.

Optionally, at step 604, compensation may be applied to the sampled output voltage DO by the compensation circuitry 508 to account for the effect of settling after reset of the reset node NR. Additionally or alternatively, compensation may be applied in the analog domain to the output voltage VO output from the amplifier 502.

When it is determined at step 606 that the settling period ST has ended, sampling of the output voltage VO by the ADC 506 may cease until after the next reset of the reset node NR.

To determine the compensation to be applied to the sampled output voltage DO, the impulse response of the cell 200 and the measurement circuit 500 after the reset at step 602 may be considered. After the switch S1 has been opened at step 602, the impulse response of the cell 200 and the measurement circuit 500 may be modelled by the following transfer function:

$$T = \frac{VO}{Vs} = \frac{Zin}{Zs + Zin}$$

Thus, to get a correct estimate of the voltage Vs across the cell 200, the inverse Tinv of the above transfer function T may be applied by the compensation circuitry 508, i.e.:

$$Tinv = \frac{Zs + Zin}{Zin}$$

The measurement circuit 500 described above can be implemented differentially to reduce or eliminate common mode noise present due to the high impedance nature of the input of the measurement circuit 500.

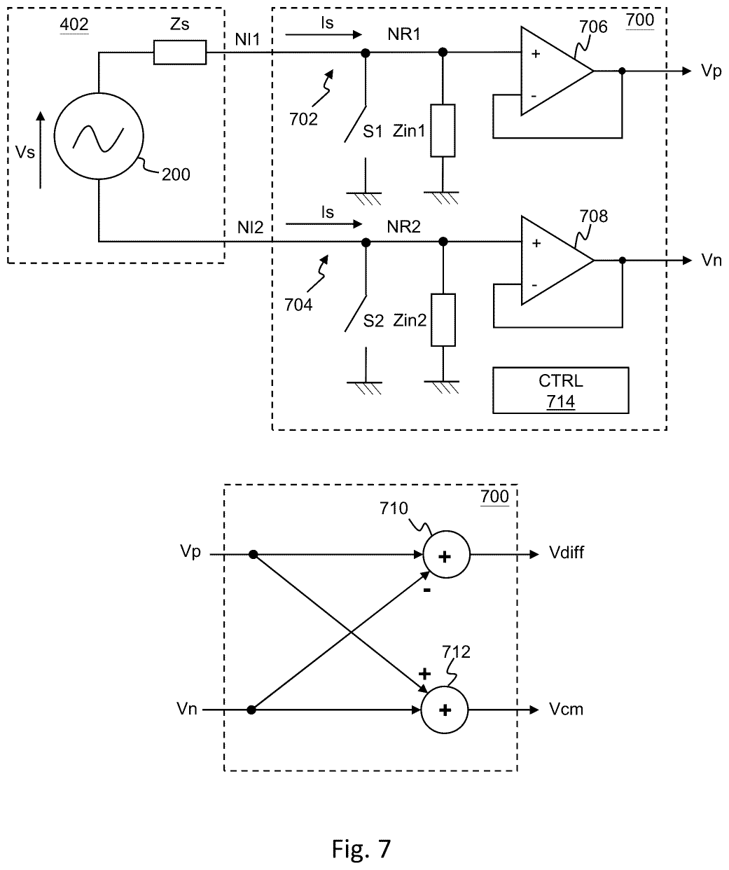

FIG. 7 is a schematic diagram of a differential measurement circuit 700 for measuring a potential difference Vs across the two-electrode cell 200 implemented as a potentiometric sensor. The measurement circuit 700 is a differential equivalent to the single ended measurement circuit 500 of FIG. 5. The equivalent circuit model 402 for the cell 200 is the same as that which is shown in FIGS. 4 and 5, hence like numbering has been applied.

The measurement circuit 700 comprises a first signal path 702 to sense a first input node NI1 coupled to a first electrode of the cell 200 and a second signal path 704 to sense a second input node NI2 coupled to a second electrode of the cell 200.

The first signal path 702 comprises a first amplifier 706, a first input impedance Zin1, and a first switches S1. The second signal path 704 comprises a second amplifier 708, a second input impedance Zin2, and a second switch S2.

A non-inverting input of the first amplifier 706 is coupled to the series impedance Zs of the cell 200. The first input impedance Zin1 is coupled between the non-inverting input of the first amplifier 706 and a reference voltage (in this case ground). An inverting input and output of the first amplifier 706 are coupled together.

A non-inverting input of the second amplifier 708 is coupled to an electrode of the cell 200 not coupled to the first amplifier 708. The second input impedance Zin2 is coupled between the non-inverting input of the second amplifier 708 and the reference voltage (in this case). An inverting input and output of the second amplifier 708 are coupled together.

Thus, like the measurement circuit 500, the measurement circuit 700 is configured as a high input impedance amplifier which buffers the sense voltage Vs across the cell 200 to the output of the measurement circuit 500 manifested as respective first and second output voltage Vp, Vn. The differential nature of the measurement circuit 700 acts to eliminate or at least ameliorate common mode noise due to the high input impedances Zin1, Zin2. The respective first and second output voltage Vp, Vn can be used to determine a differential voltage Vdiff and a common mode voltage Vcm derived from the first and second input nodes NI1, NI2. To that end, the measurement circuit 700 may further comprise circuitry for generating the differential and common mode voltage Vdiff, Vcm. Such circuitry may comprise a first summing module 710 configured to subtract the second voltage Vn from the first voltage Vp to obtain the differential voltage Vdiff, and a second summing module 712 configured to sum the first and second voltages Vn, Vp to obtain the common mode voltage Vcm.

As noted above, the measurement circuit 700 further comprises the first and second reset switches. The first reset switch S1 is coupled between a first reset node NR1 and the reference voltage (in this case ground). The first reset node NR1 is coupled to the non-inverting input of the first amplifier 706. The second reset switch S2 is coupled between a second reset node NR2 and the reference voltage (in this case ground), the second reset node NR2 coupled to the non-inverting input of the second amplifier 708. Thus, when the first reset switch S1 is closed, the first input impedance Zin1 is bypassed, and a known reference voltage is applied at the first reset node NR1. Similarly, when the second reset switch S2 is closed, the second input impedance Zin2 is bypassed, and a known reference voltage is applied at the second reset node NR2. In the example shown in FIG. 7, the same reference voltage (ground) is coupled to the first and second switches S1, S2 and the first and second impedances Zin1, Zin2. In other embodiments, the first switch S1 and first impedance Zin1 may be coupled to a different reference voltage to that coupled to the second switch S2 and second impedance Zin2.

The measurement circuit 700 further comprises control circuitry 714 (i.e. a controller) configured to control the first and second reset switches S1, S2. The control circuitry 714 may be configured to control the switches S1, S2 to reset respective first and second reset nodes NR1, NR2 periodically or in response to one or more external signals. For example, the first and second reset nodes NR1, NR2 may be reset on determination of a noise condition in the output voltage VO, such as there being too much noise in the output voltage VO or a determination that the output voltage VO inaccurately approximates the sensor voltage Vs.

A reset operation may comprise closing respective first and second reset switches S1, S2 for a predetermined time period to reset respective voltages at the first and second reset nodes NR1, NR2 to known reference voltages, followed by opening the first and second reset switches S1, S2. The control circuitry 714 may be configured to reset the first and second reset switches simultaneously. Alternatively, the control circuitry 714 may be configured to reset the first and second reset switches sequentially. For example, the control circuitry 714 may be configured to close and open the first switch S1 and then close and open the second switch S2. Staggering switching of the first and second switches S1, S2 has the advantage of avoiding shorting two terminals of the cell 200 (coupled at first and second input nodes NI1, NI2), as would be the case if both of the first and second switches S1, S2 were coupled to ground and closed at the same time. Shorting terminals of the cell 200 may be detrimental to subsequent measurements. For example, the recovery time after a reset in which terminals of the cell 200 are shorted by be in the order of hours. As such, it is preferable to avoid unnecessarily shorting terminals of the cell 200.

The control circuitry 714 may be configured to reset the first and second reset nodes NR1, NR2 immediately preceding measurement of the cell 200. Where the measurement circuit 700 is configured to measure the cell 200 periodically, the first and second reset nodes NR1, NR2 may thus be reset periodically. Additionally or alternatively, the measurement circuit 700 may be configured to measure the cell 200 in response to an external trigger. In which case, on receiving that external trigger, the control circuitry 714 may be configured to reset the first and second reset nodes NR1, NR2.

In addition to or as an alternative to the above, the control circuitry 714 may be configured to reset the first and second reset nodes NR1, NR2 based on at least one characteristic of the first and/or second voltages Vp, Vn output from the first and second amplifiers 706, 708 respectively. For example, the control circuitry 714 may monitor the common mode voltage Vcm and reset the first and/or second reset nodes NR1, NR2 in response to a drift being detecting in the common mode voltage Vcm. For example, if the common mode voltage Vcm exceeds a threshold level for longer than a predetermined time period, the control circuitry 715 may reset the first and/or second reset nodes NR1, NR2. In doing so, the measurement circuit 700 and cell 200 may be controlled so as to constrain the voltage Vs across the sensor within safe operating limits, thereby maintaining health of the cell 200.

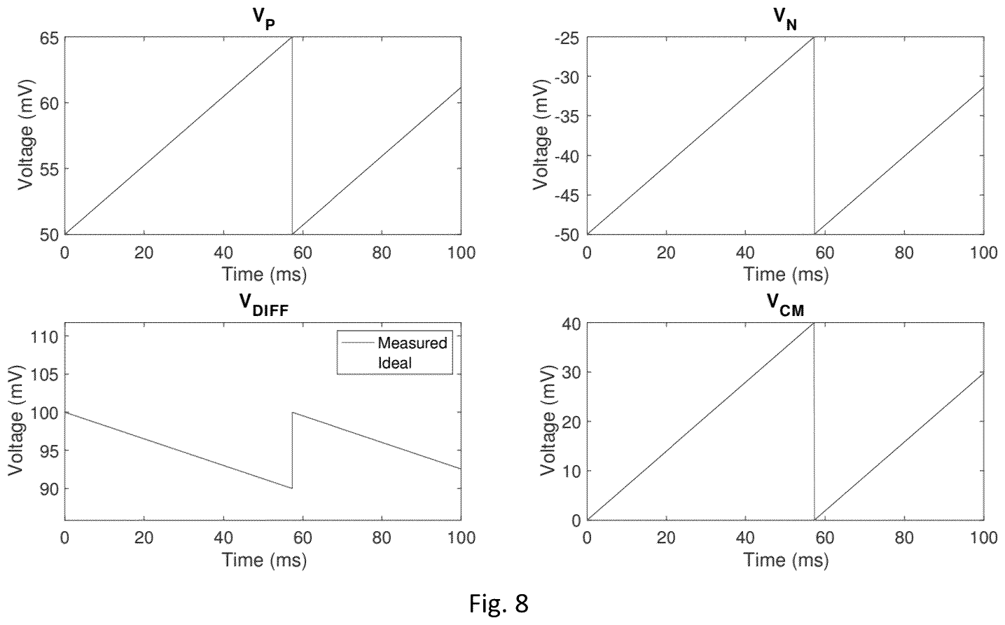

FIG. 8 graphically illustrates the first and second voltages Vp, Vn, the differential voltage Vdiff, and the common mode voltage Vcm during a reset responsive to the common mode voltage Vcm exceeding a threshold voltage VT. In this example, the threshold voltage VT is 40 mV. It can be seen that the first and second voltages Vp, Vn exhibit drift at different rates, the second voltage Vn drifting faster than the first voltage Vp. As a result, the differential voltage Vdiff (which relates to the voltage Vs across the sensor) also drifts leading to measurement error. The common mode voltage Vcm can be used as a measure of drift such that when the common mode voltage Vcm reaches the threshold voltage VT=40 mV, the first and second reset nodes NR1, NR2 are reset. In doing so, the common mode voltage Vcm returns to zero and the differential voltage Vdiff to 100 mV, which is an accurate representation of the difference between the first and second voltages Vp, Vn.

Like the measurement circuit 500, the measurement circuit 700 may further comprise further processing circuitry for sampling one or more of the first and second voltage Vp, Vn, the differential voltage Vdiff, and the common mode voltage Vcm. Such processing circuitry may comprise the analog-to-digital converter (ADC) 506 and/or compensation circuitry 508 shown in FIG. 5. Like in the circuit 500 shown in FIG. 5, compensation may be applied to compensate for the effect of settling after reset of the first and/or second reset nodes NR1, NR2. Compensation may be applied at any conceivable location in the signal chain, such as to the first and second voltages Vp, Vn, or the differential voltage Vdiff, or any digital representation thereof.

The above described solution with reference to FIG. 7 addresses DC drift in the first and second voltage Vp, Vn. It may be desirable to additionally account for AC interference, such as line noise (e.g. 50-60 Hz mains frequency), switching noise from power regulators, and the like. To address such noise, the common mode voltage Vcm may be low pass filtered. In cases where common mode drift manifests as a low frequency term, the use of a low pass filter having a cut-off frequency suitable to supress AC interference may help to isolate circuit-related drift from the presence of such noise.

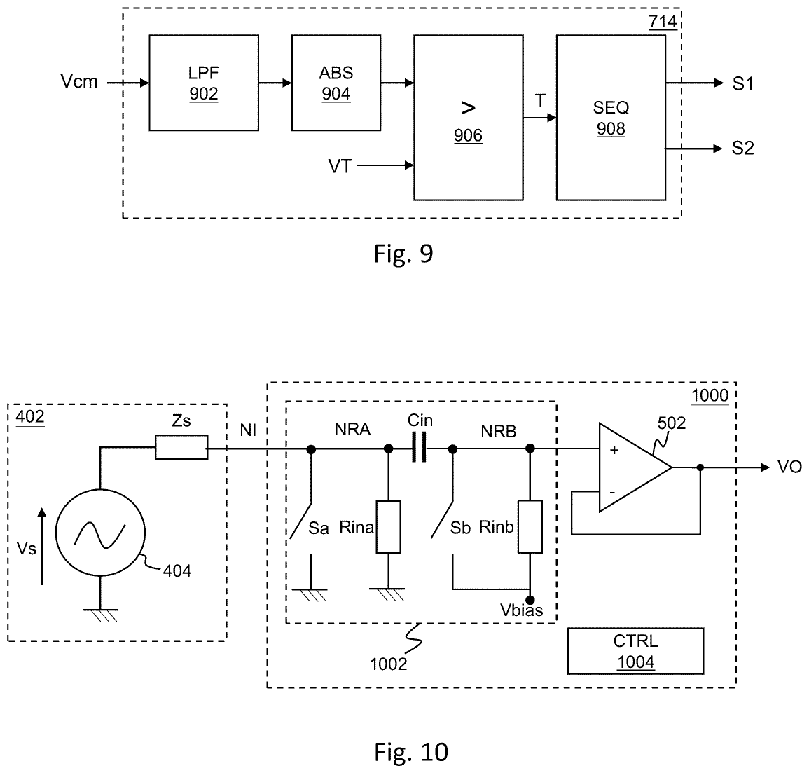

FIG. 9 is a block diagram of a block diagram of an example implementation of the control circuitry 714. The control circuitry 714 may comprise additional circuitry (not shown).

The common mode voltage Vcm is provided to a low pass filter 902 which is configured to remove AC interference. For example, the low pass filter 902 may have a cut-off frequency below 50 Hz, e.g. 40 Hz. The low pass filtered common mode voltage Vcm is passed to an absolute module 904 configured to output the absolute (or modulus) of the low-pass filtered common mode voltage Vcm. This absolute value is compared with the threshold voltage VT at a comparison module 906. When the absolute value of the low-pass filtered common mode voltage Vcm exceeds the threshold voltage, the comparison module 906 outputs a trigger signal T to a sequencer 908. In response to the trigger signal T, the sequencer 908 is configured to close and subsequently open the first and second reset switches S1, S2. Optionally, the sequencer 908 is configured to operate the first and second reset switches S1, S2 in a sequential manner. As noted above, by staggering switching of the first and second reset switches S1, S2, shorting of terminals of the cell 200 can be avoided. In some situations, it may be advantageous when resetting to include an additional phase in which the first and second switches S1, S2 are closed at the same time, thereby intentionally shorting the terminals of the cell 200. For example, where the cell 200 is potentiometric, the output voltage Vs will consist of two components as per the Nernst equation. The first component relates to the working electrode concentration, and the other term relates to the reference electrode, known as E0. The E0 term is known to drift and empirical evidence suggests that shorting the electrodes of the cell 200 resets E0 (in exchange for recovery time).

In the above measurement circuits 500, 700, the various impedances Zin, Zin1, Zin2 are provided as simply impedances coupled between respective reset nodes NR, NR1, NR2 and a reference voltage. In other embodiments, more complex impedances may be provided.

FIG. 10 provides one such example of a measurement circuit 1000 which is a variation of the circuit 500 shown in FIG. 5, like parts being denoted like numbering.

The measurement circuit 1000 differs from the measurement circuit 500 of FIG. 5 in that the reset switch S1 and input impedance Zin are replaced with a complex input impedance 1002, and the control circuitry 504 is replaced with control circuitry 1004 for controlling switches of the input impedance 1002.

The impedance 1002 comprises first and second resistors Rina, Rinb and a capacitor Cin. The capacitor Cin is coupled between a first reset node NRA (also coupled to the input node NI of the measurement circuit 1000) and a second reset node NRB coupled to the non-inverting input of the amplifier 502. The first resistor Rina is coupled between the first reset node NRA and a first reference voltage (in this case ground). The second resistor Rinb is coupled between the second reset node NRB and a second reference voltage (in this case Vbias). In some embodiments the second reference voltage Vbias is also ground.

Thus, the input impedance 1002 operates as a high pass filter which allows a time varying (or AC) component of the sense voltage Vs to pass to the amplifier 502, whilst blocking DC. This enables the non-inverting input to the amplifier 502 (the second reset node NRB) to be biased to a different DC (or time-invariant) level than the input node NI coupled to the cell 200. In doing so, the amplifier 502 may be biased by setting the second reference voltage Vbias at the second resistor Rinb so as to maximise use of the full dynamic range of the amplifier 502. As such, the amplifier 502 may no longer have to accommodate full rail-to-rail operation. In doing so, the cost and complexity of design of the amplifier 502 may be reduced. In addition, clipping associated with additional noise may be avoided by biasing the amplifier 502 approximately at the mid-point of its dynamic range. Therefore, in some embodiments, the second reference voltage Vbias may be set to Vdd/2 (i.e. half the supply voltage of the amplifier 502.

As noted above, when the cell 200 is implemented as a potentiometric sensor, the voltage Vs output from the cell 200 will be relatively small and have a relatively low frequency. In which case, it may be desirable to level shift the signal provided to the amplifier 502 to a higher voltage for downstream processing (such as by the amplifier 502, the ADC 506, and the compensation circuitry 508).

Whilst the high pass filter implemented by the impedance 1002 has the advantage of allowing the amplifier 502 to be biased to a different voltage of that at the input node NI, it will be appreciated that such filtering will affect the signal received at the input node NI of the measurement circuit 1000. Thus, it may be desirable to reverse such filtering in downstream processing. This may be achieved by applying an inverse transfer function of the high pass filter to a signal derived from the amplifier 502. Such an inverse transfer function may be implemented in either analog or digital, for example using the compensation circuitry 508. In such cases, the compensation circuitry 508 may comprise a digital signal processor (DSP) or the like to perform such processing. Additionally or alternatively, such processing may be achieved in software.

It will also be appreciated that the filtering effect of the impedance 1002 may also have an integrating effect leading to drift over time due to the presence of offsets in the measurement circuit 1000. In addition, the high pass filtering nature of the impedance 1002 leads to removal of the DC component of the signal received from the cell 200.

The inventors have realised that the combination of drift due to non-ideal effects and removal of DC signal information can be addressed with the provision of first and second reset switches Sa, Sb. Such switches Sa, Sb are provided to reset the first and second reset nodes NRA, NRB coupled to the capacitor Cin of the impedance 1002. The first reset switch Sa is coupled between the first reset node NRA and the same reference voltage to which the first resistor Rina is coupled (ground). The second reset switch Sb is coupled between the second reset node NRB and the same reference voltage Vbias to which the second resistor Rinb is coupled.

The reset switches Sa, Sb may be controlled by the control circuitry 1004 in a similar manner to the reset switch S1 of the measurement circuit 500 of FIG. 5. By resetting respective first and second reset nodes NRA, NRB to known first and second reference voltages (GND, Vbias), any drift associated with non-ideal effects in the measurement circuit 1000 can be removed. In addition, the inventors have found that the response of the impedance 1002 to the reset may be used to estimate the DC component of the sensor voltage Vs of the cell 100, despite the high pass filtering nature of the impedance 1002.

For example, during a reset of the measurement circuit 1000, the control circuitry 1004 may control the first and second reset switches Sa, Sb to close, thus resetting the first and second reset node NRA, NRB to respective first and second reference voltages GND, Vbias, after which the switches Sa, Sb may be opened.

Then, during a predetermined sampling period ST after the switches Sa, Sb have been opened, the output voltage VO may be sampled (e.g. by the ADC 506) to obtain a sampled output voltage DO. The sampling period ST may be chosen so as to approximately two orders of magnitude less than the settling time of the impedance 1002. For example, if the settling time of the impedance 1002 is in the order of seconds, the sampling period ST may be in the order of milliseconds. It will be appreciated that the effect of drift will be smallest at the point of reset. However, the longer the sampling period ST, the less noise will affect the sampled signal. Thus, a longer sampling period ST may result in increased integration drift but decreased sensitivity to noise. Conversely, a shorter sampling period ST may result in decreased integration drift but increased sensitivity to noise. The sampling period ST may be selected to trade-off the effect of offset drift versus signal noise. At the end of the sampling period ST, sampling of the output voltage VO by the ADC 506 may cease until after the next reset of the reset nodes NRA, NRB.

Compensation may be applied (e.g. by the compensation circuitry 508) to the output voltage VO, the sampled output voltage DO, or a signal derived therefrom. Such compensation may account for the effect of settling after reset of the reset nodes NRA, NRB or for the effect of filtering by the high pass filter implemented by the impedance 1002, or for both the effect of settling and the effect of filtering.

Based on the sampled output voltage DO, information about the voltage at the input node NI, such as its DC component can be obtained. For example, if multiple samples of the output voltage VO are taken over time, such samples can be fit to a curve representing the response of the output voltage VO to a reset event at a given sensor voltage Vs. In doing so, the value of Vs at the time of reset can be obtained.

Given the non-linear (e.g. exponential) response of the system, such curve fitting may be achieved using non-linear regression. However, such techniques tend neither to be computationally efficient nor robust if initial parameters used for such techniques are inadequate.

An alternative approach which is proposed herein is to first linearize the curve, either piecewise or globally, and then use linear regression to obtain an estimate. Linear regression techniques have the advantage of being more computationally efficient than non-linear regression.

As such, embodiments of the disclosure may use a closed form solution (using linear regression), as opposed to using iterative linear or non-linear least square algorithms. Doing so avoids potential excessive power consumption associated with extended searches/iterations. In using a closed form solution, an answer may be guaranteed within a given power consumption and/or time budget. This proves advantageous on embedded systems, such as those with limited power and computational resources. In addition, the accuracy of any output can be easily checked.

A further advantage of using linear regression for exponential curve fitting is that such techniques tend to be robust to noise, systematic gain error, and offsets inherent to the analogue front end (AFE) systems.

In addition to providing information regarding sensor voltage Vs, estimates of sensor response obtained using the linear regression techniques described above may also indicate other characteristics of the cell 200, such as health, by extracting quality metrics from a step or impulse response after reset of the various measurement circuits. Such quality metrics may be associated with time constants and/or oscillatory behaviour in the response.

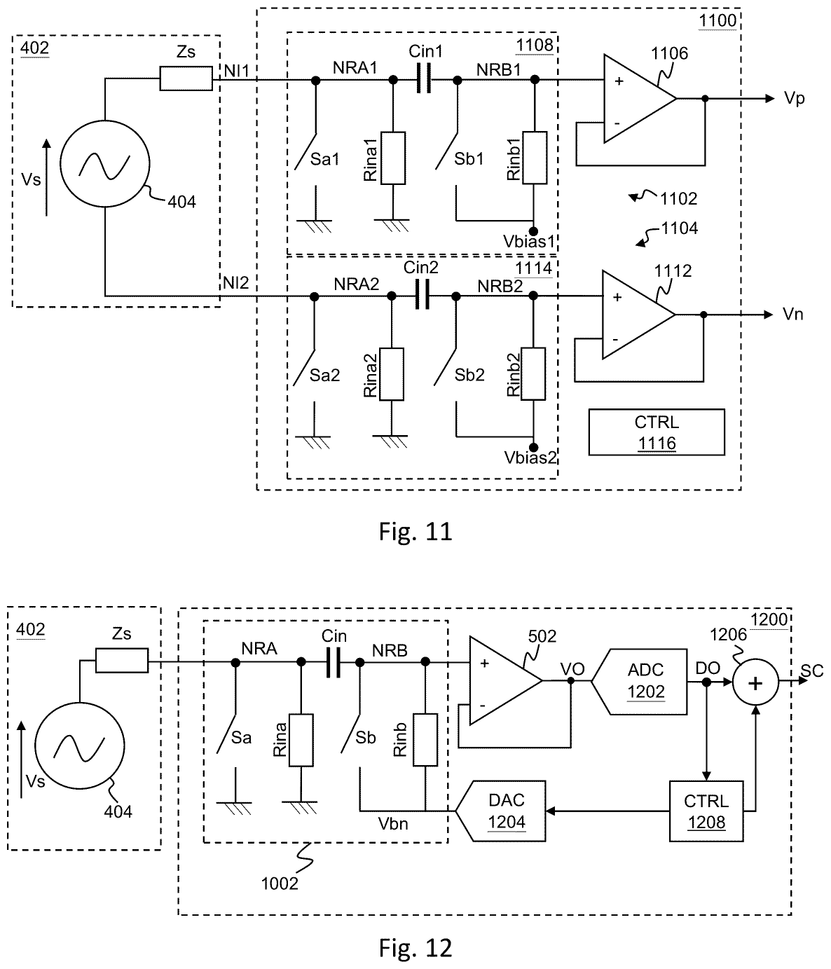

The single-ended arrangement shown in FIG. 10 may be extended to a differential arrangement as shown in FIG. 11.

FIG. 11 is a schematic diagram of a differential measurement circuit 1100 for measuring a potential difference Vs across the two-electrode cell 200 implemented as a potentiometric sensor. The measurement circuit 1100 is a differential equivalent to the single ended measurement circuit 1000 of FIG. 10 and a variation of the differential measurement circuit 700 shown in FIG. 7.

The measurement circuit 1100 comprises a first signal path 1102 to sense a first input node NI1 coupled to a first electrode of the cell 200 and a second signal path 1104 to sense a second input node NI2 coupled to a second electrode of the cell 200. Each of the first and second signal paths 1102, 1104 are equivalent to the single signal path of the measurement circuit 1000 of FIG. 10.

Specifically, the first signal path 1102 comprises a first amplifier 1106 and a first impedance 1108. The second signal path 704 comprises a second amplifier 1112 and a second impedance 1114. Each of the first and second impedances 1108, 1114 are identical to the impedance 1002 of the measurement circuit 1000 of FIG. 10.

The first impedance 1108 comprises first and second high-side resistors Rina1, Rinb1 and a high-side capacitor Cin1. The high-side capacitor Cin1 is coupled between a first high-side reset node NRA1 (also coupled to the first input node NI1 of the measurement circuit 1100) and a second high-side reset node NRB1 coupled to the non-inverting input of the first amplifier 502. The first high-side resistor Rina1 is coupled between the first high-side reset node NRA1 and a first reference voltage (in this case ground). The second high-side resistor Rinb1 is coupled between the second high-side reset node NRB1 and a second reference voltage (in this case Vbias). In some embodiments the second reference voltage Vbias is also ground.

The first impedance 1108 further comprises first and second high-side switches Sa1, Sb1. Such switches Sa1, Sb1 are provided to reset the first and second high-side reset nodes NRA1, NRB1 coupled to the high-side capacitor Cin1 of the first impedance 1108. The first reset switch Sa1 is coupled between the first high-side reset node NRA1 and the same reference voltage to which the first high-side resistor Rina1 is coupled (ground). The second high-side reset switch Sb1 is coupled between the second high-side reset node NRB1 and the same reference voltage Vbias to which the second high-side resistor Rinb1 is coupled.

The second impedance 1114 comprises first and second low-side resistors Rina2, Rinb2 and a low-side capacitor Cin2. The high-side capacitor Cin2 is coupled between a first low-side reset node NRA2 (also coupled to the second input node NI2 of the measurement circuit 1100) and a second low-side reset node NRB2 coupled to the non-inverting input of the second amplifier 1112. The first low-side resistor Rina2 is coupled between the first low-side reset node NRA2 and the first reference voltage (in this case ground). The second low-side resistor Rinb2 is coupled between the second low-side reset node NRB2 and the second reference voltage (in this case Vbias). In some embodiments the second reference voltage Vbias is also ground.

The second impedance 1114 further comprises first and second low-side switches Sa2, Sb2. Such switches Sa2, Sb2 are provided to reset the first and second low-side reset nodes NRA2, NRB2 coupled to the low-side capacitor Cin2 of the second impedance 1114. The first low-side reset switch Sa2 is coupled between the first low-side reset node NRA2 and the same reference voltage to which the first low-side resistor Rina2 is coupled (ground). The second low-side reset switch Sb2 is coupled between the second low-side reset node NRB2 and the same reference voltage Vbias to which the second low-side resistor Rinb2 is coupled.

Each of the first and second signal paths 1102, 1104 operate in a similar manner to the signal path of the measurement circuit 1000. Thus, like the measurement circuit 1000, the measurement circuit 1100 is configured as a high input impedance amplifier which buffers the sense voltage Vs across the cell 200 to the output of the measurement circuit 1100 manifested as respective first and second output voltage Vp, Vn. The differential nature of the measurement circuit 1100 acts to eliminate or at least ameliorate common mode noise due to the high input impedances 1108, 1114. The respective first and second output voltage Vp, Vn can be used to determine a differential voltage Vdiff and a common mode voltage Vcm derived from the first and second input nodes NI1, NI2. To that end, the measurement circuit 1100 may further comprise circuitry (not shown) for generating the differential and common mode voltage Vdiff, Vcm. Such circuitry is described above with reference to the measurement circuit 700 of FIG. 7.

As for the measurement circuit 1000 of FIG. 10, respective switches Sa1, Sb1, Sa2, Sb2 may be controlled by control circuitry 1116 to reset high- and low-side first and second reset nodes NRA1, NRA2 in a similar manner to that described with reference to the control circuitry 1004 of the measurement circuit 1000 of FIG. 10.

As noted above with reference to FIGS. 10 and 11, the second reference voltage Vbias can be adjusted to vary the bias at the input of respective amplifiers 502, 1106, 1112. In some embodiments, this bias voltage may be varied based on the signal output from respective amplifiers 502, 1106, 1112.

For example, FIG. 12 is a schematic diagram of a measurement circuit 1200 which is a variation of the measurement circuit 1000 of FIG. 10, like parts being denoted like numbering. The measurement circuit 1200 differs from the measurement circuit 1000 with the addition of an ADC 1202, a DAC 1204, a summing module 1206 and the control circuitry 1004 replaced with control circuitry 1208.

The ADC 1202 is coupled to the output of the amplifier 502 and is configured to convert the output voltage VO to a digital output DO representing the output voltage VO. This digital representation is provided to the control circuitry 1208 and the summing module 1206. The DAC 1204 is configured to generate the bias voltage Vbias based on a digital input signal DI from the control circuitry 1208. Based on the digital output DO, the control module 1208 may output the digital input signal DI to control the DAC 1204 to generate the bias voltage so as to optimally bias the ADC 1202 into the middle of its range. This allows the use of a higher gain before the ADC, thereby improving signal-to-noise ratio (SNR) of the digital output DO. Additionally or alternatively, the gain of the ADC 1202 can be varied by changing the reference voltage of the input stage. The control circuitry 1208 may then output a signal to the summing module to be subtracted from the digital output DO to account for the variation in bias voltage Vbias. The summing module 1206 may then output a compensated digital signal DC based on the subtraction of the signal from the control circuitry 1208 from the digital output DO.

The differential measurement circuit 1100 of FIG. 11 may be augmented in a similar manner to that shown in FIG. 12.

Embodiments are described above with reference to cells 100, 200 comprising two electrodes (e.g. a working electrode WE and a counter electrode CE, or a reference electrode RE and a working electrode WE). Embodiments of the disclosure are not, however, limited to having cells having only one counter electrode or only one working electrode. The concepts described herein are particularly applicable to cells comprising multiple working electrodes or multiple counter electrodes. In doing so, such sensors may either providing redundancy or enabling the sensing of multiple analytes in a single chip. This may be particularly advantageous in applications such as continuous glucose monitoring, where it may be desirable to measure concentrations of several analytes including but not limited to two or more of glucose, ketones, oxygen, lactate, and the like. Moreover, the measurement circuits described herein may be configurable in different configurations for different types of measurements. Such measurements may be of the same or different cells or electrodes.

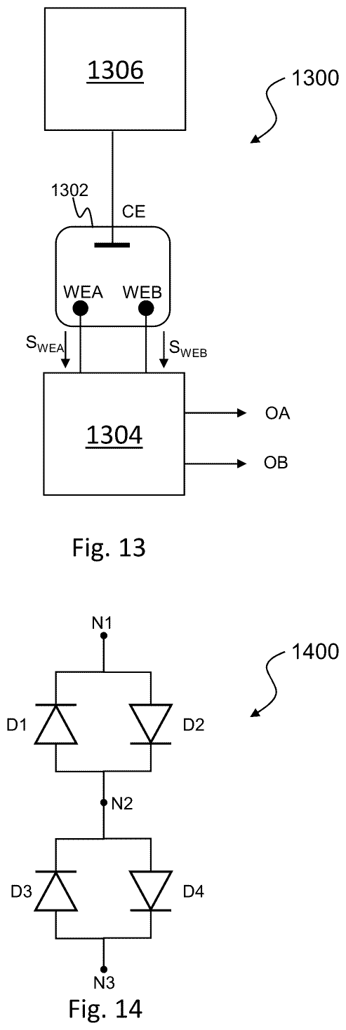

FIG. 13 illustrates an example measurement circuit 1300. In the circuit 1300, an electrochemical cell 1302 is shown comprising first and second working electrode WEA, WEB, and a counter electrode CE. A measurement circuit 1304 is provided which is configured to output a first output signal S1 based on a signal S_{WEA} derived from the first working electrode WEA and output a second output signal S2 based on a signal S_{WEB} derived from the second working electrode WEB. The measurement circuit 1304 may, for example, comprise two processing channels, each processing channel implementing the circuitry descried with reference to any of FIGS. 5 to 12. Alternatively, various components of the circuitry described above may be shared between the two processing channels, e.g., through multiplexing or similar known techniques. Optionally, drive circuitry 1306 may be provided to drive the cell 1302 with a stimulus (for example if the cell 1302 is a potentiostat).

As is described in detail above, very large input impedances are present in the measurement circuits 400, 500, 700, 1000, 1100, 1200, 1300. For some applications, such impedances are required to be in order of gigaohms or teraohms. To synthesize such resistance, embodiments of the present disclosure may utilize polysilicon diode structures.

FIG. 14 is a schematic diagram of a polysilicon diode 1400 which may be implemented as any one of the impedances described herein, such as the input impedance Zin of the measurement circuit 500 in FIG. 5. The polysilicon diode 1400 comprises first and second nodes NI, N2, and first, second, third and fourth diodes D1, D2, D3, D4. A cathode of the first diode D1 is coupled to the first node NI. An anode of the first diode D1 is coupled to the second node N2. A cathode of the second diode D2 is coupled to the second node N2 and an anode of the second diode D2 is coupled to the first node NI. A cathode of the third diode D3 is coupled to the second node N2. An anode of the third diode D3 is coupled to the third node N3. A cathode of the fourth diode D4 is coupled to the third node N3 and an anode of the fourth diode D4 is coupled to the second node N2. By providing a series combination of the first and second diodes D1, D2 in series with third and fourth diodes D3, D4, the impedance of the polysilicon diode 1400 is increase when compared to having just two diodes in parallel (e.g. just first and second diodes D1, D2).

The polysilicon diode 1400 may replace any one or more of the impedances of the circuits 400, 500, 700, 1000, 1100, 1200, 1300 described above by coupling the polysilicon diode 1400 (using first and third nodes NI, N3) in place of a respective impedance. Further examples of polysilicon diodes are described in UK patent publication number GB2466643A, the contents of which is hereby incorporated by reference in its entirety.

In a variation of the polysilicon diode 1400, the second node N2 may be omitted, such that the first and third diode D1, D3 are connected in series between the first and third nodes NI, N3 and the second and fourth diodes D2, D4 are connected in series between the first and third nodes NI, N3.

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus the code may comprise conventional program code or microcode or, for example code for setting up or controlling an ASIC or FPGA. The code may also comprise code for dynamically configuring re-configurable apparatus such as re-programmable logic gate arrays. Similarly the code may comprise code for a hardware description language such as Verilog™ or VHDL (Very high speed integrated circuit Hardware Description Language). As the skilled person will appreciate, the code may be distributed between a plurality of coupled components in communication with one another. Where appropriate, the embodiments may also be implemented using code running on a field-(re)programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general purpose processor or the like. A module may itself comprise other modules or functional units. A module may be provided by multiple components or sub-modules which need not be co-located and could be provided on different integrated circuits and/or running on different processors.

Embodiments may be implemented in a host device, especially a portable and/or battery powered host device such as a mobile computing device for example a laptop or tablet computer, a games console, a remote control device, a home automation controller or a domestic appliance including a domestic temperature or lighting control system, a toy, a machine such as a robot, an audio player, a video player, or a mobile telephone for example a smartphone.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. Circuitry for processing an analyte signal obtained from an electrochemical cell,
the circuitry comprising:
a first input node for receiving the analyte signal;
measurement circuitry having a first signal path coupled to the first input node, the measurement circuitry configured to output an output signal based on the analyte signal, the measurement circuitry comprising a first circuit element having a first impedance, the first circuit element coupled to the first signal path; and
control circuitry configured to reset one or more first voltages in the first signal path of the measurement circuitry to a respective first reference value.

2. Circuitry of claim 1, wherein the control circuitry is configured to reset the one or more voltages in the signal path based on the output signal.

3. Circuitry of claim 2, wherein the output signal is a differential output signal, and wherein the control circuitry is configured to reset the one or more voltages in the signal path based on a common mode of the differential output signal.

4. (canceled)

5. Circuitry of claim 1, wherein the one or more first voltages comprise a plurality of first voltages at different nodes of the first signal path, wherein the control circuitry is configured to reset each of the plurality of first voltages sequentially.

6. (canceled)

7. Circuitry of claim 1, wherein:
the circuitry comprises a second input node, the analyte signal received between the first and second input nodes;
the measurement circuitry further comprises:
a second signal path coupled to the second input node; and
a second circuit element having a second impedance, the second circuit element coupled to the second signal path; and
wherein the control circuitry is configured to reset one or more second voltages in the second signal path to a respective second reference value.

8. Circuitry of claim 7, wherein the control circuitry is configured to sequentially reset the one or more first voltages and the one or more second voltages or simultaneously reset the one or more first voltages and the one or more second voltages.

9. (canceled)

10. Circuitry of claim 1, further comprising processing circuitry configured to:
sample the output signal during a first sampling period after reset of the respective voltage to generate a sampled output signal; and
determine a characteristic of the analyte signal based on the sampled output signal.

11. Circuitry of claim 10, wherein the processing circuitry comprises an analog-to-digital converter (ADC), wherein simultaneous to resetting the one or more voltages in the signal path, the control circuitry is configured to reset the ADC.

12. (canceled)

13. Circuitry of claim 10, wherein the processing circuitry is configured to:
obtain a plurality of samples of the output signal during the first sampling period;
fit the plurality of samples to a predetermined curve; and
determine the characteristic of the analyte signal based on the predetermined curve.

14. Circuitry of claim 10, wherein the processing circuitry is configured to:
apply compensation to the sampled output signal to compensate for a response of the measurement circuitry to the reset of the one or more first voltages in the first signal path or to compensation for a filtering characteristic of measurement circuitry.

15.-16. (canceled)

17. Circuitry of claim 10, wherein the processing circuitry is configured to apply compensation to the sampled output signal to compensate for a response of the electrochemical cell to the reset of the one or more first voltages.

18. Circuitry of claim 10, wherein the first circuit element comprises an input filter configured to filter the analyte signal to generate a filtered analyte signal, wherein reset of the one or more first voltages comprises resetting charge on a capacitor of the input filter to a known charge.

19.-20. (canceled)

21. Circuitry of claim 18, wherein the measurement circuitry comprises a gain stage configured to apply a gain to the filtered analyte signal to obtain the output signal.

22. Circuitry of claim 7, wherein:
the first circuit element comprises a first input filter configured to filter the analyte signal to generate a first filtered analyte signal and a first gain stage configured to apply a first gain to the first filtered analyte signal to obtain a first output signal;
the second circuit element comprises a second input filter configured to filter the analyte signal to generate a second filtered analyte signal and a second gain stage configured to apply a second gain to the second filtered analyte signal to obtain a second output signal; and
the first and second output signals are combined to obtain a combined output signal.

23. (canceled)

24. Circuitry of claim 22, further comprising:
an analog-to-digital converter (ADC) configured to sample the combined output signal to obtain a sampled output signal; and
compensation circuitry configured to apply compensation to the sampled output signal based on filter characteristics of the first and second input filters, wherein the ADC comprises an incremental sigma-delta ADC, wherein the control circuitry is configured to reset the ADC when resetting the one or more first voltages in the first signal path.

25. (canceled)

26. Circuitry of claim 22, further comprising:
a first ADC configured to sample the first output signal to obtain a first sampled output signal;
a second ADC configured to sample the second output signal to obtain a second sampled output signal;
compensation circuitry configured to:
apply compensation to the first sampled output signal based on a first filter characteristic of the first filter; and
apply compensation to the second sampled output signal based on a second filter characteristic of the second filter.

27. Circuitry of claim 1, wherein the circuit element comprises one or more diodes, wherein the one or more diodes comprise silicon diodes or polysilicon diodes.

28.-29. (canceled)

30. Circuitry for processing an analyte signal obtained from an electrochemical cell, the circuitry comprising:
a first input node for receiving the analyte signal;
measurement circuitry having a first signal path coupled to the first input node, the measurement circuitry configured to output an output signal based on the analyte signal, the measurement circuitry comprising a first impedance comprising a polysilicon diode coupled to the first signal path; and
control circuitry configured to reset one or more first voltages in the first signal path of the measurement circuitry to a respective first reference value.

31. An integrated circuit (IC), comprising:
the circuitry of claim 1.

32.-33. (canceled)

34. A system comprising:
the circuitry of claim 1; and
the electrochemical cell, wherein:
the electrochemical cell comprises a counter electrode and a first working electrode.

35.-37. (canceled)

38. An electronic device, comprising the circuitry of claim 1, wherein wherein the electronic device comprises one of an analyte monitoring device or an analyte sensing device, a battery, a battery monitoring device, a mobile computing device, a laptop computer, a tablet computer, a games console, a remote control device, a home automation controller or a domestic appliance, a toy, a robot, an audio player, a video player, or a mobile telephone, and a smartphone.

39. (canceled)
